(19) United States
(12) Patent Application Publication
(10) Pub. No.: US 2020/0170992 A1
(43) Pub. Date: Jun. 4, 2020

(54) TREATMENT OF HYPERPLASTIC TISSUE GROWTHS INCLUDING BENIGN PROSTATIC HYPERPLASIA (BPH) BY DIRECT INJECTION OF AN ANTINEOPLASTIC AGENT

(71) Applicant: DFB PHARMACEUTICALS, LLC, FORT WORTH, TX (US)

(72) Inventors: GERE DIZEREGA, FORT WORTH, TX (US); H. PAUL DORMAN, FORT WORTH, TX (US)

(21) Appl. No.: 16/629,265

(22) PCT Filed: Jul. 5, 2018
(86) PCT No.: PCT/US2018/040934

§ 371 Date: Jan. 7, 2020

Related U.S. Application Data
(60) Provisional application No. 62/529,624, filed on Jul. 7, 2017.

(51) Int. Cl. A61K 31/337; A61K 9/00; A61K 9/10; A61P 13/08

(52) U.S. Cl. CPC A61K 31/337; A61P 13/08; A61K 9/10; A61K 9/0019

(57) ABSTRACT

Disclosed herein are methods for treating hyperplastic tissue growths, including benign prostatic hyperplasia, in a subject by injection of compositions comprising antineoplastic agents, including taxanes such as paclitaxel and docetaxel, directly into the hyperplastic tissue. The antineoplastic agents can be in solution in the compositions or in the form of particles dispersed in the compositions.

### CROSS-REFERENCE TO RELATED APPLICATION

This application claims priority to U.S. Provisional Application No. 62/529,624, filed Jul. 7, 2017, which is incorporated by reference herein in its entirety.

### FIELD OF THE INVENTION

The present invention generally relates to treatment of hyperplastic tissues including benign prostatic hyperplasia (BPH).

### BACKGROUND OF THE INVENTION

Hyperplasia is an increase in the number of cells in an organ or tissue due to cell proliferation and can lead to the enlargement of an organ and/or form a hyperplastic tissue growth (mass). Examples of hyperplastic tissue growths include benign prostatic hyperplasia, endometrial hyperplasia, and atypical hyperplasia of the breast.

Benign prostatic hyperplasia (BPH) is an enlarged prostate condition in males. BPH is a condition that occurs in many men as they age and causes various urinary problems. BPH is a benign condition. Symptoms include a weak urine flow, a feeling that the bladder has not emptied after urinating, a sudden urgent feeling to urinate, dribbling of urine, starting and stopping during urination, trouble starting urination, frequent need to urinate during sleep, and the need to push or strain while urinating. In some instances, BPH can cause the bladder to be blocked, making it difficult to urinate or unable to urinate. This can lead to bladder infections or kidney damage.

Treatment of BPH has included oral medications, minimally invasive surgeries, and/or major surgical procedures. Oral medications include alpha blockers and 5-alpha reductase inhibitors, and combinations thereof; however, these drugs can have side effects such as dizziness, lightheadedness, fatigue, and difficulty ejaculating.

Minimally invasive surgeries for BPH include High Intensity Focused Ultrasound (HIFU), Holmium Laser Enucleation of Prostate (HoLEP), Interstitial Laser Coagulation (ILC), Transurethral Electroevaporation of The Prostate (TUVP), Transurethral Microwave Thermotherapy (TUMT), Transurethral Needle Ablation (TUNA), Photoselective Vaporization (PVP), UroLift, and catheterization. However, men who have had a minimally invasive surgery may be at a higher risk for needing another surgery in the future. Side effects from minimally invasive surgeries can include erectile dysfunction issues, retrograde ejaculation, urinary tract infections, blood in the urine, burning sensation when urinating, needing to urinate more often, and sudden urges to urinate. Another minimally invasive procedure is placement of a prostatic stent. However, side effects from a prostatic stent include incontinence, having to urinate often, dribbling of urine, discomfort and light bleeding. Stones can also form on the stent.

Major surgical procedures for BPH include transurethral resection of the prostate (TURP), Transurethral Incision of the Prostate (TUIP), open simple prostatectomy, and radical prostatectomy. Risks of TURP can include temporary difficulty urinating, urinary tract infection, dry orgasm, erectile dysfunction, heavy bleeding, difficulty holding urine, low sodium in the blood, and need for retreatment. Risks of TUIP can include temporary difficulty urinating, urinary tract infection, and need for retreatment. Risks of radical prostatectomy include urinary tract infection, urinary incontinence, erectile dysfunction, injury to the rectum, narrowing (stricture) of the urethra or bladder neck, and formation of cysts containing lymph (lymphocele). Risks of open simple prostatectomy include heavy bleeding, urinary tract infection, urinary incontinence, dry orgasm, erectile dysfunction, and narrowing (stricture) of the urethra or bladder neck. As with any surgery that involves anesthesia, there are additional risks due to the anesthesia.

Endometrial hyperplasia is a thickening in the lining of the womb due to abnormal growth of endometrial and stromal cells. There are several types of endometrial hyperplasia and include simple hyperplasia, complex hyperplasia, simple atypical hyperplasia, and complex atypical hyperplasia. Symptoms of endometrial hyperplasia include abnormal vaginal bleeding. Endometrial hyperplasia has been treated with progestin, however treatment with progestin can cause vaginal bleeding. Other treatments include hysterectomy, which is a major surgical procedure.

Atypical hyperplasia of the breast is a precancerous condition that is an accumulation of abnormal cells in the breast. There are two types of atypical hyperplasia of the breast. Atypical ductal hyperplasia (ADH) is found within the ducts and atypical lobular hyperplasia (ALH) is found within the breast lobules. Treatments include surgical excision of the affected tissue which includes the associated risks of surgery.

### SUMMARY OF THE INVENTION

The present invention provides solutions to the aforementioned limitations and deficiencies in the art relating to treatment of hyperplastic tissue growths, including benign prostatic hyperplasia (BPH), by injection of a composition comprising an antineoplastic agent, such as a taxane, directly into the hyperplastic tissue growth.

In one aspect of the invention, disclosed is a method for treating a hyperplastic tissue growth in a subject, the method comprising injecting a composition comprising an effective amount of an antineoplastic agent directly into the hyperplastic tissue, thereby treating the hyperplastic tissue growth. In some embodiments, the composition comprises a carrier which can be an aqueous carrier and/or a liquid carrier. In some embodiments, the anti-neoplastic agent is in solution (dissolved) in the composition or carrier. In other embodiments, the anti-neoplastic agent is present as particles dispersed or suspended in the composition or carrier. In some embodiments, the particles of the antineoplastic agent have a mean particle size (number) of from 0.1 microns to 10 microns, or from 0.1 microns to 5 microns, or from 0.1 microns to 1.5 microns, or from 0.1 microns to 1 micron, or from 0.1 microns to less than 1 micron. In preferred embodiments, the antineoplastic agent is a taxane. In further preferred embodiments, the taxane is paclitaxel or docetaxel. In some various embodiments, the hyperplastic tissue is benign prostatic hyperplasia (BPH), endometrial hyperplasia, or atypical hyperplasia of the breast. In some embodiments, the hyperplastic tissue growth is reduced in volume/size, or has a reduced growth rate after injecting the composition.

In another aspect of the invention, disclosed is a method for treating benign prostatic hyperplasia (BPH) in a subject, the method comprising injecting a composition comprising an effective amount of a taxane directly into the prostate of the subject (intraprostatic injection), thereby treating the BPH. In some embodiments, the composition is injected into at least one lateral lobe of the prostate. In some embodiments, the composition is injected into the left lateral lobe and the right lateral lobe of the prostate. In some embodiments, the taxane is in solution (dissolved) in the composition. In other embodiments, the taxane is present as particles dispersed or suspended in the composition. In some embodiments, the taxane particles have a mean particle size (number) of from 0.1 microns to 10 microns, or from 0.1 microns to 5 microns, or from 0.1 microns to 1.5 microns, or from 0.1 microns to 1 micron, or from 0.1 microns to less than 1 micron. In preferred embodiments, the taxane is paclitaxel or docetaxel. In some embodiments, the prostate is reduced in volume/size, and/or has a reduced growth rate after injecting the composition into the prostate. In some embodiments, the subject experiences an improvement of urinary problems associated with BPH after injection the composition into the prostate.

Also, disclosed in the context of the present invention are the following embodiments 1 to 28:
Embodiment 1 is a method for treating a hyperplastic tissue growth in a subject, the method comprising injecting a composition comprising a carrier an effective amount of an antineoplastic agent directly into the hyperplastic tissue growth, thereby treating the hyperplastic tissue growth.
Embodiments 2 is the method of embodiment 1, wherein the carrier is a liquid carrier.
Embodiments 3 is the method of any one of embodiments 1 to 2, wherein the carrier is an aqueous carrier.
Embodiments 4 is the method of any one of embodiments 1 to 3, wherein the antineoplastic agent is dissolved in the carrier.
Embodiments 5 is the method of any one of embodiments 1 to 3, wherein the antineoplastic agent is the form of particles suspended in the carrier.
Embodiments 6 is the method of embodiment 5, wherein the antineoplastic particles have a mean particle size (number) of from 0.1 microns to 10 microns, or from 0.1 microns to 5 microns, or from 0.1 microns to 1.5 microns, or from 0.1 microns to 1 microns, or from 0.1 microns to less than 1 micron.
Embodiments 7 is the method of any one of embodiments 1 to 6, wherein the antineoplastic agent is a taxane.
Embodiments 8 is the method of embodiment 7, wherein the taxane is paclitaxel or docetaxel.
Embodiments 9 is the method of any one of embodiments 1 to 8, wherein the injection of the composition is conducted by endoscopic ultrasound-guided fine needle injection (EUS-FNI).
Embodiments 10 is the method of any one of embodiments 1 to 9, wherein the hyperplastic tissue growth is benign prostatic hyperplasia (BPH), endometrial hyperplasia, or atypical hyperplasia of the breast.
Embodiments 11 is the method of any one of embodiments 1 to 10, wherein the hyperplastic tissue growth is reduced in volume/size, or has a reduced growth rate after injecting the composition.
Embodiments 12 is a method of treating benign prostatic hyperplasia (BPH) in a subject, the method comprising injecting a composition comprising a carrier an effective amount of a taxane directly into the prostate of the subject, thereby treating the BPH.
Embodiments 13 is the method of embodiment 12, wherein the carrier is a liquid carrier.
Embodiments 14 is the method of any one of embodiments 12 to 13, wherein the carrier is an aqueous carrier.
Embodiments 15 is the method of any one of embodiments 12 to 14, wherein the taxane is dissolved in the carrier.
Embodiments 16 is the method of embodiment 15, wherein the taxane is paclitaxel or docetaxel.
Embodiments 17 is the method of any one of embodiments 12 to 14 wherein the taxane is the form of particles suspended in the carrier.
Embodiments 18 is the method of embodiment 17, wherein the taxane particles have a mean particle size (number) of from 0.1 microns to 10 microns, or from 0.1 microns to 5 microns, or from 0.1 microns to 1.5 microns, or from 0.1 microns to 1 microns, or from 0.1 microns to less than 1 micron.
Embodiments 19 is the method of any one of embodiments 17 to 18, wherein the taxane particles are paclitaxel particles or docetaxel particles.
Embodiments 20 is the method of embodiment 19, wherein the paclitaxel particles or docetaxel particles have a specific surface area (SSA) of at least 18 m²/g.
Embodiments 21 is the method of embodiment 19, wherein the paclitaxel particles or docetaxel particles have a specific surface area (SSA) of less than 18 m²/g.
Embodiments 22 is the method of any one of embodiments 19 to 21, wherein the paclitaxel particles or docetaxel particles have a bulk density (not-tapped) of 0.05 g/cm³ to 0.15 g/cm³.
Embodiments 23 is the method of any one of embodiments 19 to 21, wherein the paclitaxel particles or docetaxel particles have a bulk density (not-tapped) of greater than 0.15 g/cm³, or greater than 0.20 g/cm³.
Embodiments 24 is the method of any one of embodiments 19 to 23, wherein the concentration of paclitaxel particles or docetaxel particles in the composition is between about 6 mg/mL and about 15 mg/mL.
Embodiments 25 is the method of any one of embodiments 12 to 24, wherein the composition is injected into at least one lateral lobe of the prostate.
Embodiments 26 is the method of embodiment 25, wherein the composition is injected into the left lateral lobe and the right lateral lobe of the prostate.
Embodiments 27 is the method of any one of embodiments 12 to 26, wherein the injection of the composition is conducted by endoscopic ultrasound-guided fine needle injection (EUS-FNI).
Embodiments 28 is the method of any one of embodiments 12 to 27, wherein the prostate is reduced in volume/size, or has a reduced growth rate after injecting the composition, and/or the subject experiences an improvement in urinary problems associated with BPH.

The term “antineoplastic agents” as used herein are drugs used to treat neoplasms including non-cancerous neoplasms and malignant neoplasms, and include “chemotherapeutic agents”, which are drugs used to treat cancer. In a preferred embodiment, the antineoplastic agent is a taxane.

The terms “antineoplastic agent particles”, “antineoplastic particles” or “particles of an antineoplastic agent”, as used herein are particles of an antineoplastic agent and have a mean particle size (number) of from about 0.1 microns to about 10 microns (about 100 nm to about 10,000 nm) in diameter. In a preferred embodiment, the antineoplastic particles are taxane particles.

The term “benign prostatic hyperplasia” (BPH) means and an enlarged prostate condition in a subject, wherein the enlargement is due to hyperplastic benign cell proliferation.

As used herein, the terms “treat”, “treatment”, “treated”, or “treating” with respect to a hyperplastic tissue growth means accomplishing one or more of the following: (a) reducing the hyperplastic tissue growth volume/size; (b) reducing the rate of growth of the hyperplastic tissue growth.

As used herein, the terms “treat”, “treatment”, “treated”, or “treating” with respect to BPH means accomplishing one or more of the following: (a) reducing the prostate volume/size; (b) reducing the prostate growth rate.

As used herein, the terms “suspension” or “dispersion” mean a suspension dosage form composition where antineoplastic particles are dispersed (suspended) within a continuous carrier or a continuous carrier/diluent mixture. In a suspension or dispersion, the antineoplastic particles can be completely dispersed, partially dispersed and partially dissolved, but not completely dissolved in the carrier or carrier/diluent mixture.

The terms “subject” or “patient” as used herein mean a vertebrate animal. In some embodiments, the vertebrate animal can be a mammal. In some embodiments, the mammal can be a primate, including a human. In some embodiments with respect to treatment of BPH, the subject is a human male.

The term “room temperature” (RT) as used herein, means 20-25° C.

The term “surfactant” or “surface active agent” as used herein, means a compound or a material or a substance that exhibits the ability to lower the surface tension of water or to reduce the interfacial tension between two immiscible substances.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

For this application, a number value with one or more decimal places can be rounded to the nearest whole number using standard rounding guidelines, i.e. round up if the number being rounded is 5, 6, 7, 8, or 9; and round down if the number being rounded is 0, 1, 2, 3, or 4. For example, 3.7 can be rounded to 4.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The use of the word “a” or “an” when used in conjunction with the terms “comprising,” “having,” “including,” or “containing” (or any variations of these words) may mean “one,” but it is also consistent with the meaning of “one or more,” “at least one,” and “one or more than one.”

The compositions and methods for their use can “comprise,” “consist essentially of,” or “consist of any of” the ingredients or steps disclosed throughout the specification. With respect to the phrase “consisting essentially of,” a basic and novel property of the compositions of the present invention are their ability to topically treat hyperplastic tissue growths including BPH.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION OF THE INVENTION

In one aspect of the invention, disclosed, is are methods for treating hyperplastic tissue growths in a subject, the method comprising injecting a composition comprising an effective amount of an antineoplastic agent directly into the hyperplastic tissue, thereby treating the hyperplastic tissue growth. In some embodiments, the composition comprises a carrier which can be an aqueous carrier and/or a liquid carrier. In some embodiments, the anti-neoplastic agent is in solution (dissolved) in the composition or carrier. In other embodiments, the anti-neoplastic agent is present as particles dispersed or suspended in the composition or carrier. In some embodiments, the particles of the antineoplastic agent have a mean particle size (number) of from 0.01 microns to 10 microns. In some embodiments, the particles of the antineoplastic agent have a mean particle size (number) of from 0.1 microns to 10 microns, or from 0.1 microns to 5 microns, or from 0.1 microns to 1.5 microns, or from 0.1 microns to 1 micron, or from 0.1 microns to less than 1 micron. In preferred embodiments, the antineoplastic agent is a taxane. In further preferred embodiments, the taxane is paclitaxel or docetaxel. In some various embodiments, the hyperplastic tissue is benign prostatic hyperplasia (BPH), endometrial hyperplasia, or atypical hyperplasia of the breast. In some embodiments, the hyperplastic tissue growth is reduced in volume/size, or has a reduced growth rate after treatment by injection of the composition.

In another aspect of the invention, disclosed are methods for treating benign prostatic hyperplasia (BPH) in a subject, the methods comprising injecting a composition comprising an effective amount of a taxane directly into the prostate of the subject (intraprostatic injection), thereby treating the BPH. In some embodiments, the composition is injected into at least one lateral lobe of the prostate. In some embodiments, the composition is injected into the left lateral lobe and the right lateral lobe of the prostate. In some embodiments, the taxane is in solution (dissolved) in the composition. In other embodiments, the taxane is present as particles dispersed or suspended in the composition. In some embodiments, the taxane particles have a mean particle size (number) of from 0.01 microns to 10 microns. In some embodiments, the taxane particles have a mean particle size (number) of from 0.1 microns to 10 microns, or from 0.1 microns to 5 microns, or from 0.1 microns to 1.5 microns, or from 0.1 microns to 1 micron, or from 0.1 microns to less than 1 micron. In preferred embodiments, the taxane is paclitaxel or docetaxel. In some embodiments, the enlarged prostate is reduced in volume/size, and/or has a reduced growth rate after treatment by injection of the composition.

Antineoplastic Agents and Antineoplastic Agent Particles

Antineoplastic agents are drugs used to treat neoplasms including malignant, pre-cancerous, and non-malignant (benign) neoplasms, and include “chemotherapeutic agents”, which are drugs used to treat cancer. Non-limiting examples of antineoplastic agents include taxanes such as paclitaxel, derivatives of paclitaxel, docetaxel, cabazitaxel; epothilones; Vinca alkaloids such as vinblastine, vincristine, vindesine, vinorelbine; camptothecins such as topotecan; platinum complexes such as cisplatin, carboplatin, oxaliplatin; podophyllotoxins such as etoposide and teniposide; and 5-fluorouracil. Other non-limiting examples of antineoplastic agents can be found listed in the “Ashgate Handbook of Antineoplastic Agents”, published by Gower Publishing Limited, 2000, herein incorporated by reference. The antineoplastic agent can be in solution in the compositions or can be in the form of particles suspended in the compositions. The antineoplastic agent particles can have a mean particle size (number) of from about 0.01 microns (10 nm) to about 10 microns (10,000 microns) in diameter, or from about 0.1 microns to about 10 microns (about 100 nm to about 10,000 nm) in diameter, or from about 0.1 microns to about 5 microns in diameter (about 100 nm to about 5000 nm), or from about 0.1 microns to about 1.5 microns (about 100 nm to about 1500 nm) in diameter, or from about 0.1 microns to about 1 micron (about 100 nm to about 1000 nm) in diameter, or from about 0.1 microns to less than 1 micron (about 100 nm to less than 1000 nm) in diameter.

In some embodiments, the antineoplastic particles are solid, uncoated (“neat” or “naked”) individual particles. In some embodiments, the antineoplastic particles are not bound to any substance. In some embodiments, no substances are absorbed or adsorbed onto the surface of the antineoplastic particles. In some embodiments, the antineoplastic agents or antineoplastic particles are not encapsulated, contained, enclosed or embedded within any substance. In some embodiments, the antineoplastic particles are not coated with any substance. In some embodiments, the antineoplastic particles are not microemulsions, nanoemulsions, microspheres, or liposomes containing an antineoplastic agent. In some embodiments, the antineoplastic particles are not bound to, encapsulated in, or coated with a monomer, a polymer (or biocompatible polymer), a protein, a surfactant, or albumin. In some embodiments, a monomer, a polymer (or biocompatible polymer), a protein, a surfactant, or albumin is not absorbed or adsorbed onto the surface of the antineoplastic particles. In some embodiments, the antineoplastic particles are not clusters or agglomerates of individual antineoplastic particles.

In some embodiments, the antineoplastic particles are in crystalline form. In other embodiments, the antineoplastic particles are in amorphous form, or a combination of both crystalline and amorphous form. In some embodiments, the antineoplastic particles of the invention contain traces of impurities and byproducts typically found during preparation of the antineoplastic agent. In some embodiments, the antineoplastic particles comprise at least 90%, at least 95%, at least 96%, at least 97%, at least 98%, at least 99% or 100% of the antineoplastic agent, meaning the antineoplastic particles consist of or consist essentially of substantially pure antineoplastic agent.

In some embodiments, the antineoplastic particles are coated with or bound to a substance such as a protein (e.g., albumin), a monomer, a polymer, a biocompatible polymer, or a surfactant. In some embodiments, a substance such as a protein (e.g., albumin), a monomer, a polymer, a biocompatible polymer, or a surfactant is adsorbed or absorbed onto the surface of the antineoplastic particles. In some embodiments, the antineoplastic particles are encapsulated, contained, enclosed, or embedded within a substance such as a protein (e.g., albumin), a monomer, a polymer, a biocompatible polymer, or a surfactant. In some embodiments, the antineoplastic particles are microemulsions, nanoemulsions, microspheres, or liposomes containing an antineoplastic agent. In some embodiments, the antineoplastic particles are clusters or agglomerates of individual antineoplastic particles.

In preferred embodiments, the antineoplastic agent is a taxane. Taxanes are poorly water-soluble compounds. Poorly water-soluble compounds generally have a solubility of less than or equal to 10 mg/mL in water at room temperature. Taxanes are widely used as antineoplastic agents and chemotherapy agents. The term “taxanes” as used herein include paclitaxel (I), docetaxel (II), cabazitaxel (III), and any other taxane or taxane derivatives, non-limiting examples of which are taxol B (cephalomannine), taxol C, taxol D, taxol E, taxol F, taxol G, taxadiene, baccatin III, 10-deacetylbaccatin, taxchinin A, brevifoliol, and taxuspine D, and also include pharmaceutically acceptable salts of taxanes.

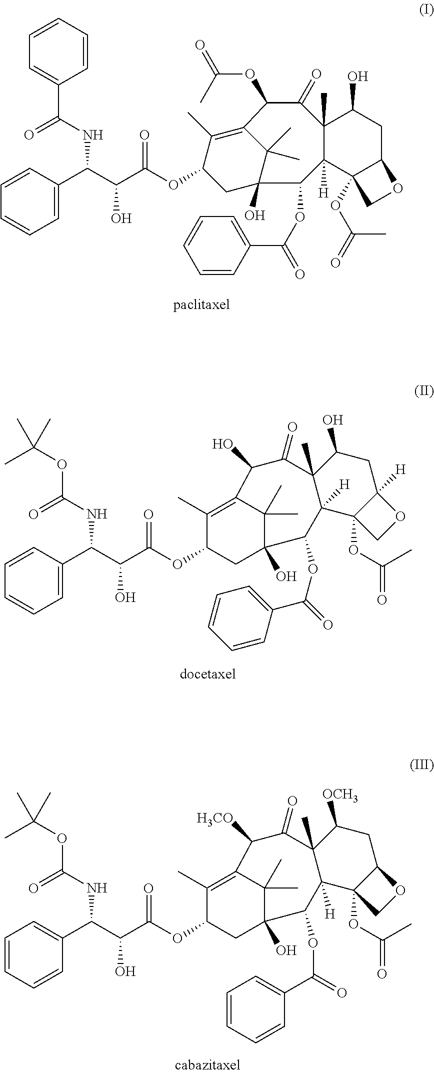

Paclitaxel and docetaxel active pharmaceutical ingredients (APIs) are commercially available from Phyton Biotech LLC, Vancouver, Canada. The docetaxel API contains not less than 90%, or not less than 95%, or not less than 97.5% docetaxel calculated on the anhydrous, solvent-free basis. The paclitaxel API contains not less than 90%, or not less than 95%, or not less than 97% paclitaxel calculated on the anhydrous, solvent-free basis. In some embodiments, the paclitaxel API and docetaxel API are USP and/or EP grade. Paclitaxel API can be prepared from a semisynthetic chemical process or from a natural source such as plant cell fermentation or extraction. Paclitaxel is also sometimes referred to by the trade name TAXOL™, although this is a misnomer because TAXOL is the trade name of a solution of paclitaxel in polyoxyethylated castor oil and ethanol intended for dilution with a suitable parenteral fluid prior to intravenous infusion. Taxane APIs can be used to make taxane particles. In some embodiments, the antineoplastic particles are taxane particles. The taxane particles can be paclitaxel particles, docetaxel particles, or cabazitaxel particles, or particles of other taxane derivatives, including particles of pharmaceutically acceptable salts of taxanes.

Taxane particles can have a mean particle size (number) of from about 0.01 microns to about 10 microns (about 10 nm to about 10,000 nm) in diameter, or from about 0.1 microns to about 10 microns (about 100 nm to about 10,000 nm) in diameter, or from about 0.1 microns to about 5 microns in diameter (about 100 nm to about 5000 nm), or from about 0.1 microns to about 1.5 microns (about 100 nm to about 1500 nm) in diameter, or from about 0.1 microns to about 1 micron (about 100 nm to about 1000 nm) in diameter, or from about 0.1 microns to less than 1 micron (about 100 nm to less than 1000 nm) in diameter.

In some embodiments, the taxane particles are solid, uncoated (neat) individual particles. In some embodiments, the taxane particles are not bound to any substance. In some embodiments, no substances are absorbed or adsorbed onto the surface of the taxane particles. In some embodiments, the taxane or taxane particles are not encapsulated, contained, enclosed or embedded within any substance. In some embodiments, the taxane particles are not coated with any substance. In some embodiments, the taxane particles are not microemulsions, nanoemulsions, microspheres, or liposomes containing a taxane. In some embodiments, the taxane particles are not bound to, encapsulated in, or coated with a monomer, a polymer (or biocompatible polymer), a protein, a surfactant, or albumin. In some embodiments, a monomer, a polymer (or biocompatible polymer), a protein, a surfactant, or albumin is not absorbed or adsorbed onto the surface of the taxane particles. In some embodiments, the taxane particles exclude albumin. In some embodiments, the taxane particles are paclitaxel particles and exclude albumin. In some embodiments, the taxane particles are not clusters or agglomerates of individual taxane particles.

In some embodiments, the taxane particles are in crystalline form. In other embodiments, the taxane particles are in amorphous form, or a combination of both crystalline and amorphous form. In some embodiments, the taxane particles of the invention contain traces of impurities and byproducts typically found during preparation of the taxane. In some embodiments, the taxane particles comprise at least 90%, at least 95%, at least 96%, at least 97%, at least 98%, at least 99% or 100% of the taxane, meaning the taxane particles consist of or consist essentially of substantially pure taxane.

In some embodiments, the taxane particles are coated with or bound to a substance such as a protein (e.g., albumin), a monomer, a polymer, a biocompatible polymer, or a surfactant. In some embodiments, a substance such as a protein (e.g., albumin), a monomer, a polymer, a biocompatible polymer, or a surfactant is adsorbed or absorbed onto the surface of the taxane particles. In some embodiments, the taxane particles are encapsulated, contained, enclosed, or embedded within a substance such as a protein (e.g., albumin), a monomer, a polymer, a biocompatible polymer, or a surfactant. In some embodiments, the taxane particles are microemulsions, nanoemulsions, microspheres, or liposomes containing a taxane. In some embodiments, the taxane particles are clusters or agglomerates of individual taxane particles.

The antineoplastic particles or taxane particles (including paclitaxel particles, docetaxel particles, or cabazitaxel particles) can have a mean particle size (number) of from 0.01 microns to 10 microns, or from 0.01 microns to 9 microns, or from 0.01 microns to 8 microns, or from 0.01 microns to 7 microns, or from 0.01 microns to 6 microns, or from 0.01 microns to 5 microns, or from 0.01 microns to 2 microns, or from 0.01 microns to 1.5 microns, or from 0.01 microns to 1.2 microns, or from 0.01 microns to 1 micron, or from 0.01 microns to less than 1 micron or from 0.1 microns to 10 microns, or from 0.1 microns to 9 microns, or from 0.1 microns to 8 microns, or from 0.1 microns to 7 microns, or from 0.1 microns to 6 microns, or from 0.1 microns to 5 microns, or from 0.1 microns to 2 microns, or from 0.1 microns to 1.5 microns, or from 0.1 microns to 1.2 microns, or from 0.1 microns to 1 micron, or from 0.1 microns to less than 1 micron, or from 0.1 microns to 0.9 microns, or from 0.1 microns to 0.8 microns, or from 0.1 to 0.7 microns, or from 0.2 microns to 5 microns, or from 0.2 microns to 2 microns, or from 0.2 microns to 1.5 microns, or from 0.2 microns to 1.2 microns, or from 0.2 microns to 1 micron, or from 0.2 microns to less than 1 micron, or from 0.2 microns to 0.9 microns, or from 0.2 microns to 0.8 microns, or from 0.2 microns to 0.7 microns, or from 0.3 microns to 5 microns, or from 0.3 microns to 2 microns, or from 0.3 microns to 1.5 microns, or from 0.3 microns to 1.2 microns, or from 0.3 microns to 1 micron, or from 0.3 microns to less than 1 micron, or from 0.3 microns to 0.9 microns, or from 0.3 microns to 0.8 microns, or from 0.3 microns to 0.7 microns, or from 0.4 microns to 5 microns, or from 0.4 microns to 2 microns, or from 0.4 microns to 1.5 microns, or from 0.4 microns to 1.2 microns, or from 0.4 microns to 1 micron, or from 0.4 microns to less than 1 micron, or from 0.4 microns to 0.9 microns, or from 0.4 microns to 0.8 microns, or from 0.4 microns to 0.7 microns, or from 0.5 microns to 5 microns, or from 0.5 microns to 2 microns, or from 0.5 microns to 1.5 microns, or from 0.5 microns to 1.2 microns, or from 0.5 microns to 1 micron, or from 0.5 microns to less than 1 micron, or from 0.5 microns to 0.9 microns, or from 0.5 microns to 0.8 microns, or from 0.5 microns to 0.7 microns, or from 0.6 microns to 5 microns, or from 0.6 microns to 2 microns, or from 0.6 microns to 1.5 microns, or from 0.6 microns to 1.2 microns, or from 0.6 microns to 1 micron, or from 0.6 microns to less than 1 micron, or from 0.6 microns to 0.9 microns, or from 0.6 microns to 0.8 microns, or from 0.6 microns to 0.7 microns.

The particle size of the antineoplastic particles including taxane particles can be determined by a particle size analyzer instrument and the measurement is expressed as the mean diameter based on a number distribution (number). A suitable particle size analyzer instrument is one which employs the analytical technique of light obscuration, also referred to as photozone or single particle optical sensing (SPOS). A suitable light obscuration particle size analyzer instrument is the ACCUSIZER, such as the ACCUSIZER 780 SIS, available from Particle Sizing Systems, Port Richey, Fla. Another suitable particle size analyzer instrument is one which employs laser diffraction, such as the Shimadzu SALD-7101.

Antineoplastic agent particles including taxane particles can be manufactured using various particle size-reduction methods and equipment known in the art. Such methods include, but are not limited to conventional particle size-reduction methods such as wet or dry milling, micronizing, disintegrating, and pulverizing. Other methods include “precipitation with compressed anti-solvents” (PCA) such as with supercritical carbon dioxide. Suitable antineoplastic and/or taxane particles can be made by PCA methods as disclosed in U.S. Pat. Nos. 5,874,029, 5,833,891, 6,113,795, 7,744,923, 8,778,181, 9,233,348; US publications US 2015/0375153, US 2016/0354336, US 2016/0374953; and international patent application publications WO 2016/197091, WO 2016/197100, and WO 2016/197101; all of which are herein incorporated by reference.

In PCA particle size reduction methods using supercritical carbon dioxide, supercritical carbon dioxide (anti-solvent) and solvent, e.g. acetone or ethanol, are employed to generate uncoated antineoplastic or taxane particles as small as 0.1 to 5 microns within a well-characterized particle-size distribution. The carbon dioxide and solvent are removed during processing (up to 0.5% residual solvent may remain), leaving antineoplastic or taxane particles as a powder. As disclosed in US publication 2016/0374953, herein incorporated by reference, taxane particles (e.g., paclitaxel and docetaxel) produced by PCA methods can have physical characteristics that include a bulk density (not tapped) between 0.05 g/cm³ and 0.15 g/cm³ and a specific surface area (SSA) of at least 18 m²/g of taxane particles. This bulk density range is generally lower than the bulk density of taxane particles produced by conventional means, and the SSA is generally higher than the SSA of taxane particles produced by conventional means. As used herein, the “specific surface area” (SSA) is the total surface area of the taxane particle per unit of taxane mass as measured by the Brunauer-Emmett-Teller (“BET”) isotherm by the following method: a known mass between 200 and 300 mg of the analyte is added to a 30 mL sample tube. The loaded tube is then mounted to a Porous Materials Inc. SORPTOMETER®, model BET-202A. The automated test is then carried out using the BETWIN® software package and the surface area of each sample is subsequently calculated. The BET specific surface area test procedure is a compendial method included in both the United States Pharmaceopeia and the European Pharmaceopeia. The bulk density measurement can be conducted by pouring the taxane particles into a graduated cylinder without tapping at room temperature, measuring the mass and volume, and calculating the bulk density.

As disclosed in US publication 2016/0374953, studies showed a SSA of 15.0 m²/g and a bulk density of 0.31 g/cm³ for paclitaxel particles produced by milling paclitaxel in a Deco-PBM-V-0.41 ball mill suing a 5 mm ball size, at 600 RPM for 60 minutes at room temperature. Also disclosed in US publication 2016/0374953, one lot of paclitaxel particles had a SSA of 37.7 m²/g and a bulk density of 0.085 g/cm³ when produced by a supercritical carbon dioxide method using the following method: a solution of 65 mg/mL of paclitaxel was prepared in acetone. A BETE MicroWhirl® fog nozzle (BETE Fog Nozzle, Inc.) and a sonic probe (Qsonica, model number Q700) were positioned in the crystallization chamber approximately 8 mm apart. A stainless steel mesh filter with approximately 100 nm holes was attached to the crystallization chamber to collect the precipitated paclitaxel particles. The supercritical carbon dioxide was placed in the crystallization chamber of the manufacturing equipment and brought to approximately 1200 psi at about 38° C. and a flow rate of 24 kg/hour. The sonic probe was adjusted to 60% of total output power at a frequency of 20 kHz. The acetone solution containing the paclitaxel was pumped through the nozzle at a flow rate of 4.5 mL/minute for approximately 36 hours. Additional lots of paclitaxel particles produced by the supercritical carbon dioxide method described above had SSA values of: 22.27 m²/g, 23.90 m²/g, 26.19 m²/g, 30.02 m²/g, 31.16 m²/g, 31.70 m²/g, 32.59 m²/g, 33.82 m²/g, 35.90 m²/g, 38.22 m²/g, and 38.52 m²/g.

As disclosed in US publication 2016/0374953, studies showed a SSA of 15.2 m²/g and a bulk density of 0.44 g/cm³ for docetaxel particles produced by milling docetaxel in a Deco-PBM-V-0.41 ball mill suing a 5 mm ball size, at 600 RPM for 60 minutes at room temperature. Also disclosed in US publication 2016/0374953, docetaxel particles had a SSA of 44.2 m²/g and a bulk density of 0.079 g/cm³ when produced by a supercritical carbon dioxide method using the following method: A solution of 79.32 mg/mL of docetaxel was prepared in ethanol. The nozzle and a sonic probe were positioned in the pressurizable chamber approximately 9 mm apart. A stainless steel mesh filter with approximately 100 nm holes was attached to the pressurizable chamber to collect the precipitated docetaxel particles. The supercritical carbon dioxide was placed in the pressurizable chamber of the manufacturing equipment and brought to approximately 1200 psi at about 38° C. and a flow rate of 68 slpm. The sonic probe was adjusted to 60% of total output power at a frequency of 20 kHz. The ethanol solution containing the docetaxel was pumped through the nozzle at a flow rate of 2 mL/minute for approximately 95 minutes). The precipitated docetaxel particles were then collected from the supercritical carbon dioxide as the mixture is pumped through the stainless steel mesh filter. The filter containing the particles of docetaxel was opened and the resulting product was collected from the filter.

As disclosed in US publication 2016/0374953, dissolution studies showed an increased dissolution rate in methanol/water media of paclitaxel and docetaxel particles made by the supercritical carbon dioxide methods described in US publication 2016/0374953 as compared to paclitaxel and docetaxel particles made by milling paclitaxel and docetaxel using a Deco-PBM-V-0.41 ball mill suing a 5 mm ball size, at 600 RPM for 60 minutes at room temperature. The procedures used to determine the dissolution rates are as follows. For paclitaxel, approximately 50 mg of material were coated on approximately 1.5 grams of 1 mm glass beads by tumbling the material and beads in a vial for approximately 1 hour. Beads were transferred to a stainless steel mesh container and placed in the dissolution bath containing methanol/water 50/50 (v/v) media at 37° C., pH 7, and a USP Apparatus II (Paddle), operating at 75 rpm. At 10, 20, 30, 60, and 90 minutes, a 5 mL aliquot was removed, filtered through a 0.22 μm filter and analyzed on a UV/VIS spectrophotometer at 227 nm. Absorbance values of the samples were compared to those of standard solutions prepared in dissolution media to determine the amount of material dissolved.

For docetaxel, approximately 50 mg of material was placed directly in the dissolution bath containing methanol/water 15/85 (v/v) media at 37° C., pH 7, and a USP Apparatus II (Paddle), operating at 75 rpm. At 5, 15, 30, 60, 120 and 225 minutes, a 5 mL aliquot was removed, filtered through a 0.22 μm filter, and analyzed on a UV/VIS spectrophotometer at 232 nm. Absorbance values of the samples were compared to those of standard solutions prepared in dissolution media to determine the amount of material dissolved. For paclitaxel, the dissolution rate was 47% dissolved in 30 minutes for the particles made by the supercritical carbon dioxide method versus 32% dissolved in 30 minutes for the particles made by milling. For docetaxel, the dissolution rate was 27% dissolved in 30 minutes for the particles made by the supercritical carbon dioxide method versus 9% dissolved in 30 minutes for the particles made by milling.

In some embodiments, the antineoplastic particles, including taxane particles, have a SSA of at least 10, at least 12, at least 14, at least 16, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 m²/g. In one embodiment, the antineoplastic particles, including taxane particles, have an SSA of between about 10 m²/g and about 50 m²/g. In some embodiments, the antineoplastic particles, including taxane particles, have an SSA of less than 18 m²/g, or less than 17 m²/g, or less than 16 m²/g, or less than 15 m²/g, or less than 14 m²/g, or less than 13 m²/g, or less than 12 m²/g, or less than 11 m²/g, or less than 10 m²/g. In some embodiments, the antineoplastic particles, including taxane particles, have an SSA of from 5 m²/g to less than 18 m²/g, or from 5 m²/g to 17 m²/g, or from 1 m²/g to less than 18 m²/g, or from 1 m²/g to 17 m²/g.

In some embodiments, the antineoplastic particles, including taxane particles, have a bulk density (not tapped) between about 0.050 g/cm³ and about 0.20 g/cm³. In other embodiments, the antineoplastic particles, including taxane particles, have a bulk density of greater than 0.15 g/cm³, or greater than 0.20 g/cm³. In other embodiments, the antineoplastic particles, including taxane particles, have a bulk density of from greater than 0.15 g/cm³ to 0.50 g/cm³, or from greater than 0.20 g/cm³ to 0.50 g/cm³, or from 0.25 g/cm³ to 0.50 g/cm³.

In some embodiments, the taxane particles are paclitaxel particles and have an SSA of at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35 m²/g. In other embodiments, the paclitaxel particles have an SSA of 18 m²/g to 50 m²/g, or 20 m²/g to 50 m²/g, or 22 m²/g to 50 m²/g, or 25 m²/g to 50 m²/g, or 26 m²/g to 50 m²/g, or 30 m²/g to 50 m²/g, or 35 m²/g to 50 m²/g, or 18 m²/g to 45 m²/g, or 20 m²/g to 45 m²/g, or 22 m²/g to 45 m²/g, or 25 m²/g to 45 m²/g, or 26 m²/g to 45 m²/g or 30 m²/g to 45 m²/g, or 35 m²/g to 45 m²/g, or 18 m²/g to 40 m²/g, or 20 m²/g to 40 m²/g , or 22 m²/g to 40 m²/g, or 25 m²/g to 40 m²/g, or 26 m²/g to 40 m²/g, or 30 m²/g to 40 m²/g, or 35 m²/g to 40 m²/g. In other embodiments, the paclitaxel particles have an SSA of less than 18 m²/g, or less than 17 m²/g, or less than 16 m²/g, or less than 15 m²/g, or less than 14 m²/g, or less than 13 m²/g, or less than 12 m²/g, or less than 11 m²/g, or less than 10 m²/g. In some embodiments, the paclitaxel particles have an SSA of from 5 m²/g to less than 18 m²/g, or from 5 m²/g to 17 m²/g, or from 1 m²/g to less than 18 m²/g, or from 1 m²/g to 17 m²/g.

In some embodiments, the paclitaxel particles have a bulk density (not-tapped) of 0.05 g/cm³ to 0.15 g/cm³, or 0.05 g/cm³ to 0.20 g/cm³. In other embodiments, the paclitaxel particles have a bulk density of greater than 0.15 g/cm³, or greater than 0.20 g/cm³. In other embodiments, the paclitaxel particles have a bulk density of from greater than 0.15 g/cm³ to 0.50 g/cm³, or from greater than 0.20 g/cm³ to 0.50 g/cm³, or from 0.25 g/cm³ to 0.50 g/cm³.

In some embodiments, the paclitaxel particles have a dissolution rate of at least 40% w/w dissolved in 30 minutes or less in a solution of 50% methanol/50% water (v/v) in a USP II paddle apparatus operating at 75 RPM, at 37° C., and at a pH of 7. In other embodiments, the paclitaxel particles have a dissolution rate of less than 40% w/w, or less than 35% w/w, dissolved in 30 minutes in a solution of 50% methanol/50% water (v/v) in a USP II paddle apparatus operating at 75 RPM, at 37° C., and at a pH of 7.

In some embodiments, the taxane particles are docetaxel particles and have an SSA of at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, or at least 42 m²/g. In other embodiments, the docetaxel particles have an SSA of 18 m²/g to 60 m²/g, or 22 m²/g to 60 m²/g, or 25 m²/g to 60 m²/g, or 30 m²/g to 60 m²/g, or 40 m²/g to 60 m²/g, or 18 m²/g to 50 m²/g, or 22 m²/g to 50 m²/g, or 25 m²/g to 50 m²/g, or 26 m²/g to 50 m²/g, or 30 m²/g to 50 m²/g, or 35 m²/g to 50 m²/g, or 40 m²/g to 50 m²/g. In other embodiments, the docetaxel particles have an SSA of less than 18 m²/g, or less than 17 m²/g, or less than 16 m²/g, or less than 15 m²/g, or less than 14 m²/g, or less than 13 m²/g, or less than 12 m²/g, or less than 11 m²/g, or less than 10 m²/g. In some embodiments, the docetaxel particles have an SSA of from 5 m²/g to less than 18 m²/g, or from 5 m²/g to 17 m²/g, or from 1 m²/g to less than 18 m²/g, or from 1 m²/g to 17 m²/g.

In some embodiments, the docetaxel particles have a bulk density (not-tapped) of 0.05 g/cm³ to 0.15 g/cm³. In other embodiments, the docetaxel particles have a bulk density of greater than 0.15 g/cm³, or greater than 0.20 g/cm³. In other embodiments, the docetaxel particles have a bulk density of from greater than 0.15 g/cm³ to 0.50 g/cm³, or from greater than 0.20 g/cm³ to 0.50 g/cm³, or from 0.25 g/cm³ to 0.50 g/cm³.

In some embodiments, the docetaxel particles have a dissolution rate of at least 20% w/w dissolved in 30 minutes or less in a solution of 15% methanol/85% water (v/v) in a USP II paddle apparatus operating at 75 RPM, at 37° C., and at a pH of 7. In other embodiments, the docetaxel particles have a dissolution rate of less than 20% w/w, or less than 15% w/w dissolved in 30 minutes in a solution of 15% methanol/85% water (v/v) in a USP II paddle apparatus operating at 75 RPM, at 37° C., and at a pH of 7.

The antineoplastic particles, including taxane particles, can be packaged into any suitable container such as glass or plastic vials. A non-limiting example of a suitable container is a Type 1, USP, clear-glass vial closed with a bromobutyl rubber stopper and aluminum crimp seal. The antineoplastic particles can be sterilized after the particles are in the container using sterilization methods known in the art such as gamma irradiation or autoclaving.

Compositions

The compositions of the invention comprise an antineoplastic agent, such as a taxane. In some embodiments, the compositions comprise a carrier and an antineoplastic agent. In some embodiments, the antineoplastic agent is dissolved in the carrier. In preferred embodiments, the antineoplastic agent dissolved in the carrier is a taxane, such as paclitaxel or docetaxel. Suitable compositions that are solutions of taxanes include TAXOL™ (paclitaxel) Injection which is a solution of paclitaxel in polyoxyethylated castor oil and dehydrated alcohol, and TAXOTERE™ (docetaxel) Injection which is a solution of docetaxel in polysorbate 80. In other embodiments, the antineoplastic agent is in the form of particles and is dispersed (suspended) in the carrier. In preferred embodiments, the antineoplastic particles are taxane particles, such as paclitaxel particles or docetaxel particles.

The carrier can be a liquid (fluid) carrier. The carrier can be an aqueous carrier. Non-limiting examples of suitable aqueous carriers include water, such as Sterile Water for Injection USP; 0.9% saline solution (normal saline), such as 0.9% Sodium Chloride for Injection USP; dextrose solution, such as 5% Dextrose for Injection USP; and Lactated Ringer's Solution for Injection USP. Non-aqueous based liquid carriers, such as ethyl alcohol or surfactants can be used. Other aqueous-based liquid carriers can be used. The carrier can be a pharmaceutically acceptable carrier, i.e., suitable for administration to a subject by injection or other routes of administration. The carrier can be any other type of liquid such as emulsions or flowable semi-solids. Non-limiting examples of flowable semisolids include gels and thermosetting gels. The composition can be a solution, i.e., wherein all the components including the antineoplastic agent are dissolved and in solution. The composition can be a suspension, i.e., a suspension dosage form composition where the antineoplastic particles, such as taxane particles, are dispersed (suspended) within a continuous carrier/and or diluent. In a suspension composition, the antineoplastic particles can be completely dispersed, partially dispersed and partially dissolved, but not completely dissolved in the carrier. In some embodiments, the composition is a solution of a taxane. In some embodiments, the composition is a suspension of taxane particles dispersed within a continuous carrier. In a preferred embodiment, the carrier is a pharmaceutically acceptable carrier. In preferred embodiments, the composition is sterile. In various embodiments, the composition comprises, consists essentially of, or consists of antineoplastic particles and a liquid carrier, wherein the composition is a suspension of the antineoplastic particles dispersed within the liquid carrier. In some embodiments, the composition consists essentially of or consists of antineoplastic particles and a carrier, wherein the carrier is an aqueous carrier and wherein the composition is a suspension.

The composition of a carrier and an antineoplastic agent or antineoplastic particles can be administered as-is. Optionally, the composition can further comprise a suitable diluent to dilute the composition in order to achieve a desired concentration (dose) of the antineoplastic agent or antineoplastic particles. In some embodiments, the carrier can serve as the diluent; stated another way, the amount of carrier in the composition provides the desired concentration of antineoplastic agent or antineoplastic particles in the composition and no further dilution is needed. A suitable diluent can be a fluid, such as an aqueous fluid. Non-limiting examples of suitable aqueous diluents include water, such as Sterile Water for Injection USP; 0.9% saline solution (normal saline), such as 0.9% Sodium Chloride for Injection USP; dextrose solution, such as 5% Dextrose for Injection USP; and Lactated Ringer's Solution for Injection USP. Other liquid and aqueous-based diluents suitable for administration by injection can be used and can optionally include salts, buffering agents, and/or other excipients. In some embodiments, the diluent is sterile. The composition can be diluted with the diluent at a ratio to provide a desired concentration dosage of the antineoplastic agent or antineoplastic particles. For example, the volume ratio of composition to diluent might be in the range of 1:1-1:100 v/v or other suitable ratios. In some embodiments, the composition comprises an antineoplastic agent, a carrier, and a diluent, wherein the carrier and diluent form a mixture, and wherein the antineoplastic agent is dissolved in the carrier/diluent mixture. In some embodiments, the composition comprises antineoplastic particles, a carrier, and a diluent, wherein the carrier and diluent form a mixture, and wherein the composition is a suspension of antineoplastic particles dispersed in the carrier/diluent mixture. In some embodiments, the carrier/diluent mixture is a continuous phase and the antineoplastic particles are a dispersed phase.

The composition, carrier, and/or diluent can further comprise functional ingredients such as buffers, salts, osmotic agents, surfactants, viscosity modifiers, rheology modifiers, suspending agents, pH adjusting agents such as alkalinizing agents or acidifying agents, tonicity adjusting agents, preservatives, antimicrobial agents including quaternary ammonium compounds such as benzalkonium chloride and benzethonium chloride, demulcents, antioxidants, antifoaming agents, chelating agents, and/or colorants. For example, the composition can comprise taxane particles and a carrier comprising water, a salt, a surfactant, and optionally a buffer. In some embodiments, the carrier is an aqueous carrier and comprises a surfactant, wherein the concentration of the surfactant is from about 0.01% v/v to about 50% v/v. In other embodiments, the aqueous carrier excludes the surfactants GELUCIRE® (polyethylene glycol glycerides composed of mono-, di- and triglycerides and mono- and diesters of polyethylene glycol) and/or CREMOPHOR® (polyethoxylated castor oil). In some embodiments, the composition or carrier excludes polymers, proteins (such as albumin), polyethoxylated castor oil, and/or polyethylene glycol glycerides composed of mono-, di- and triglycerides and mono- and diesters of polyethylene glycol. In other embodiments, the aqueous carrier includes the surfactants GELUCIRE® (polyethylene glycol glycerides composed of mono-, di- and triglycerides and mono- and diesters of polyethylene glycol) and/or CREMOPHOR® (polyethoxylated castor oil). In some embodiments, the composition or carrier includes polymers, proteins (such as albumin), polyethoxylated castor oil, and/or polyethylene glycol glycerides composed of mono-, di- and triglycerides and mono- and diesters of polyethylene glycol.

The composition, carrier, and/or diluent can comprise one or more surfactants. Suitable surfactants include by way of example and without limitation polysorbates, lauryl sulfates, acetylated monoglycerides, diacetylated monoglycerides, and poloxamers, such as poloxamer 407. Polysorbates are polyoxyethylene sorbitan fatty acid esters which are a series of partial fatty acid esters of sorbitol and its anhydrides copolymerized with approximately 20, 5, or 4 moles of ethylene oxide for each mole of sorbitol and its anhydrides. Non-limiting examples of polysorbates are polysorbate 20, polysorbate 21, polysorbate 40, polysorbate 60, polysorbate 61, polysorbate 65, polysorbate 80, polysorbate 81, polysorbate 85, and polysorbate 120. Polysorbates containing approximately 20 moles of ethylene oxide are hydrophilic nonionic surfactants. Examples of polysorbates containing approximately 20 moles of ethylene oxide include polysorbate 20, polysorbate 40, polysorbate 60, polysorbate 65, polysorbate 80, polysorbate 85, and polysorbate 120. Polysorbates are available commercially from Croda under the tradename TWEEN™. The number designation of the polysorbate corresponds to the number designation of the TWEEN, e.g., polysorbate 20 is TWEEN 20, polysorbate 40 is TWEEN 40, polysorbate 60 is TWEEN 60, polysorbate 80 is TWEEN 80, etc. USP/NF grades of polysorbate include polysorbate 20 NF, polysorbate 40 NF, polysorbate 60 NF, and polysorbate 80 NF. Polysorbates are also available in PhEur grades (European Pharmacopoeia), BP grades, and JP grades. The term “polysorbate” is a non-proprietary name. The chemical name of polysorbate 20 is polyoxyethylene 20 sorbitan monolaurate. The chemical name of polysorbate 40 is polyoxyethylene 20 sorbitan monopalmitate. The chemical name of polysorbate 60 is polyoxyethylene 20 sorbitan monostearate. The chemical name of polysorbate 80 is polyoxyethylene 20 sorbitan monooleate. In some embodiments, the composition, carrier, and/or diluent can comprise mixtures of polysorbates. In some embodiments, the composition, carrier, and/or diluent comprises polysorbate 20, polysorbate 40, polysorbate 60, polysorbate 65, polysorbate 80, polysorbate 85, and/or polysorbate 120. In some embodiments, the composition, carrier, and/or diluent comprises polysorbate 20, polysorbate 40, polysorbate 60, and/or polysorbate 80. In one embodiment, the composition, carrier, and/or diluent comprises polysorbate 80. The amount of surfactant in the carrier can range from about 0.01% v/v to about 99% v/v. In some embodiments, the composition comprises antineoplastic particles, a carrier, and optionally a diluent, wherein the carrier and/or diluent comprises water and a polysorbate. In one embodiment, the composition is a suspension, the antineoplastic particles are taxane particles, and the polysorbate is polysorbate 80.

The composition, carrier, and/or diluent can comprise one or more tonicity adjusting agents. Suitable tonicity adjusting agents include by way of example and without limitation, one or more inorganic salts, electrolytes, sodium chloride, potassium chloride, sodium phosphate, potassium phosphate, sodium, potassium sulfates, sodium and potassium bicarbonates and alkaline earth metal salts, such as alkaline earth metal inorganic salts, e.g., calcium salts, and magnesium salts, mannitol, dextrose, glycerin, propylene glycol, and mixtures thereof.

The composition, carrier, and/or diluent can comprise one or more buffering agents. Suitable buffering agents include by way of example and without limitation, dibasic sodium phosphate, monobasic sodium phosphate, citric acid, sodium citrate, tris(hydroxymethyl)aminomethane, bis(2-hydroxyethyl)iminotris-(hydroxymethyl)methane, and sodium hydrogen carbonate and others known to those of ordinary skill in the art. Buffers are commonly used to adjust the pH to a desirable range for intraperitoneal use. Usually a pH of around 5 to 9.5 to 8.6 to 7.4, 6.5 to 7.5, or 6.9 to 7.4 is desired.

The composition, carrier, and/or diluent can comprise one or more demulcents. A demulcent is an agent that forms a soothing film over a mucous membrane, such as the membranes lining the peritoneum and organs therein. A demulcent may relieve minor pain and inflammation and is sometimes referred to as a mucoprotective agent. Suitable demulcents include cellulose derivatives ranging from about 0.2 to about 2.5% such as carboxymethylcellulose sodium, hydroxyethyl cellulose, hydroxypropyl methylcellulose, and methylcellulose; gelatin at about 0.01%; polyols in about 0.05 to about 1%, also including about 0.05 to about 1%, such as glycerin, polyethylene glycol 300, polyethylene glycol 400, and propylene glycol; polyvinyl alcohol from about 0.1 to about 4%; povidone from about 0.1 to about 2%; and dextran 70 from about 0.1% when used with another polymeric demulcent described herein.

The composition, carrier, and/or diluent can comprise one or more alkalinizing agents to adjust the pH. As used herein, the term “alkalizing agent” is intended to mean a compound used to provide an alkaline medium. Such compounds include, by way of example and without limitation, ammonia solution, ammonium carbonate, potassium hydroxide, sodium carbonate, sodium bicarbonate, and sodium hydroxide and others known to those of ordinary skill in the art

The composition, carrier, and/or diluent can comprise one or more acidifying agents to adjust the pH. As used herein, the term “acidifying agent” is intended to mean a compound used to provide an acidic medium. Such compounds include, by way of example and without limitation, acetic acid, amino acid, citric acid, nitric acid, fumaric acid and other alpha hydroxy acids, hydrochloric acid, ascorbic acid, and nitric acid and others known to those of ordinary skill in the art.

The composition, carrier, and/or diluent can comprise one or more antifoaming agents. As used herein, the term “antifoaming agent” is intended to mean a compound or compounds that prevents or reduces the amount of foaming that forms on the surface of the fill composition. Suitable antifoaming agents include by way of example and without limitation, dimethicone, SIMETHICONE, octoxynol and others known to those of ordinary skill in the art.

The composition, carrier, and/or diluent can comprise one or more viscosity modifiers that increase or decrease the viscosity of the suspension. Suitable viscosity modifiers include methylcellulose, hydroxypropyl methycellulose, mannitol, polyvinylpyrrolidone, cross-linked acrylic acid polymers such as carbomer, and others known to those of ordinary skill in the art. The composition, carrier, and/or diluent can further comprise rheology modifiers to modify the flow characteristics of the composition to allow it to adequately flow through devices such as injection needles or tubes. Non-limiting examples of viscosity and rheology modifiers can be found in “Rheology Modifiers Handbook—Practical Use and Application” Braun, William Andrew Publishing, 2000.

The concentration or amount of antineoplastic agent or antineoplastic particles in the composition or dosage is at an “effective amount”, i.e., to provide a therapeutic effect on hyperplastic tissue growth of a subject by accomplishing the following: reducing hyperplastic tissue growth volume/size or reducing hyperplastic tissue growth rate. In the case of BPH treatment, the effective amount of the antineoplastic agent or antineoplastic particles in the composition accomplishes the following: the prostate is reduced in volume/size or has a reduced growth rate, and/or the subject experiences an improvement in urinary problems associated with BPH. In one embodiment, the concentration of the antineoplastic agent or antineoplastic particles, which can be a taxane or taxane particles respectively, in the composition is between about 0.1 mg/mL and about 100 mg/mL. In various further embodiments, the concentration in the composition is between: about 0.5 mg/mL and about 100 mg/mL, about 1 mg/mL and about 100 mg/mL, about 2 mg/mL and about 100 mg/mL, about 5 mg/mL and about 100 mg/mL, about 10 mg/mL and about 100 mg/mL, about 25 mg/mL and about 100 mg/mL, about 30 mg/mL and about 100 mg/mL, about 0.1 mg/mL and about 75 mg/mL, about 0.5 mg/mL and about 75 mg/mL, about 1 mg/mL and about 75 mg/mL, about 2 mg/mL and about 75 mg/mL, about 5 mg/mL and about 75 mg/mL, about 10 mg/mL and about 75 mg/mL, about 25 mg/mL and about 75 mg/mL, about 30 mg/mL and about 75 mg/mL, about 0.1 mg/mL and about 50 mg/mL, about 0.5 mg/mL and about 50 mg/mL, about 1 mg/mL and about 50 mg/mL, about 2 mg/mL and about 50 mg/mL, about 5 mg/mL and about 50 mg/mL, about 10 mg/mL and about 50 mg/mL, about 25 mg/mL and about 50 mg/mL, about 30 mg/mL and about 50 mg/mL, about 0.1 mg/mL and about 40 mg/mL, about 0.5 mg/mL and about 40 mg/mL, about 1 mg/mL and about 40 mg/mL, about 2 mg/mL and about 40 mg/mL, about 5 mg/mL and about 40 mg/mL, about 10 mg/mL and about 40 mg/mL, about 25 mg/mL and about 40 mg/mL, about 30 mg/mL and about 40 mg/mL, about 0.1 mg/mL and about 30 mg/mL, about 0.5 mg/mL and about 30 mg/mL, about 1 mg/mL and about 30 mg/mL, about 2 mg/mL and about 30 mg/mL, about 5 mg/mL and about 30 mg/mL, about 10 mg/mL and about 30 mg/mL, about 25 mg/mL and about 30 mg/mL, about 0.1 mg/mL and about 25 mg/mL, about 0.5 mg/mL and about 25 mg/mL, about 1 mg/mL and about 25 mg/mL, about 2 mg/mL and about 25 mg/mL, about 5 mg/mL and about 25 mg/mL, about 10 mg/mL and about 25 mg/mL, about 0.1 mg/mL and about 20 mg/mL, about 0.5 mg/mL and about 20 mg/mL, about 1 mg/mL and about 20 mg/mL, about 2 mg/mL and about 20 mg/mL, about 5 mg/mL and about 20 mg/mL, about 10 mg/mL and about 20 mg/mL, about 0.1 mg/mL and about 15 mg/mL, about 0.5 mg/mL and about 15 mg/mL, about 1 mg/mL and about 15 mg/mL, about 2 mg/mL and about 15 mg/mL, about 5 mg/mL and about 15 mg/mL, about 10 mg/mL and about 15 mg/mL, about 0.1 mg/mL and about 10 mg/mL, about 0.5 mg/mL and about 10 mg/mL, about 1 mg/mL and about 10 mg/mL, about 2 mg/mL and about 10 mg/mL, about 5 mg/mL and about 10 mg/mL, about 0.1 mg/mL and about 5 mg/mL, about 0.5 mg/mL and about 5 mg/mL, about 1 mg/mL and about 5 mg/mL, about 2 mg/mL and about 5 mg/mL, about 0.1 mg/mL and about 2 mg/mL, about 0.5 mg/mL and about 2 mg/mL, about 1 mg/mL and about 2 mg/mL, about 0.1 mg/mL and about 1 mg/mL, about 0.5 mg/mL and about 1 mg/mL, about 0.1 mg/mL and about 0.5 mg/mL, about 3 mg/mL and about 8 mg/mL, or about 4 mg/mL and about 6 mg/mL; or at least about 0.1, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 61, 65, 70, 75, or 100 mg/mL; or about 0.1, 0.5, 1, 2, 3, 4, 5, 6, 7, 8, 9, 10, 11, 12, 13, 14, 15, 16, 17, 18, 19, 20, 25, 30, 35, 40, 41, 42, 43, 44, 45, 46, 47, 48, 49, 50, 55, 60, 61, 65, 70, 75, or 100 mg/mL. The antineoplastic particles may be the sole therapeutic agent administered, or may be administered with other therapeutic agents.

In various embodiments, the composition comprises taxane particles (paclitaxel particles or docetaxel particles), a carrier, and a diluent, wherein the concentration of taxane particles in the composition (including the carrier and diluent) is between: about 1 mg/mL and about 40 mg/mL, about 5 mg/mL and about 20 mg/mL, about 5 mg/mL and about 15 mg/mL, about 5 mg/mL and about 10 mg/mL, about 6 mg/mL and about 20 mg/mL, about 6 mg/mL and about 15 mg/mL, about 6 mg/mL and about 10 mg/mL, about 10 mg/mL and about 20 mg/mL, or about 10 mg/mL and about 15 mg/mL; or about 6 mg/mL, about 10 mg/mL, or about 15 mg/mL. In further embodiments, the carrier is an aqueous carrier which can be saline solution, such as about 0.9% sodium chloride solution and the diluent is an aqueous diluent which can be saline solution, such as about 0.9% sodium chloride solution. In further embodiments, the aqueous carrier comprises a polysorbate, such as polysorbate 80. The compositions should be at suitable volume to supply a sufficient injectable dose volume for a given situation. For example, in the case of BPH treatment, the injection volume can be at a volume of 10% of the prostate.

Kits

The present invention also provides kits, comprising:

(a) a first vial comprising, consisting essentially of, or consisting of antineoplastic particles;

(b) a second vial comprising a pharmaceutically acceptable carrier; and

(c) instructions for reconstituting the antineoplastic particles into a suspension or solution useful for injection into hyperplastic tissue growth by: combining the contents of the first vial and the second vial to form the suspension or solution and optionally diluting the suspension or solution with a diluent.

In preferred embodiments, the antineoplastic particles are taxane particles such as paclitaxel particles or docetaxel particles. The antineoplastic particles in the first vial can be in a powder form. The antineoplastic particles in the first vial can be the sole ingredient in the first vial. In some embodiments, the taxane particles have a mean particle size (number) of from 0.1 microns to 1.5 microns. The pharmaceutically acceptable carrier can be an aqueous carrier such as 0.9% saline solution. The carrier can further comprise a surfactant such as a polysorbate. In some embodiments, the polysorbate is polysorbate 80.

Any suitable vial can be used in the kits. A non-limiting example of a suitable vial is a Type 1, USP, clear-glass vial closed with a bromobutyl rubber stopper and aluminum crimp seal. The volumes of the vials can vary depending on the amount of antineoplastic particles, the volume of the carrier, and the volume of the final reconstituted suspension or solution. The vials and their contents can be sterilized using sterilization methods known in the art such as gamma irradiation or autoclaving. In some embodiments, the contents of the vials are sterile. The kits can be configured for single-dose or multiple-dose administration.

A non-limiting exemplary procedure for preparing a suspension or solution composition from a kit is as follows:
1. Using a syringe with a suitable gauge needle, add all or a portion of the carrier from the second vial into the first vial containing the anti-neoplastic particles.
2. Vigorously hand shake the first vial with inversions to form a solution or suspension of the particles.
3. Immediately after shaking, use a syringe with a suitable gauge needle to add a prescribed volume of a diluent to the first vial to dilute the suspension or solution to a desired dose level, and hand shake the vial for another 1 minute.

The compositions, suspensions, solutions, and kits of the invention can include any embodiment or combination of embodiments described herein including any embodiments of the antineoplastic particles, any embodiments of the carriers and diluents, and any embodiments of other ingredients.

Methods of Administration/Treatment

The compositions comprising antineoplastic particles, including taxane particles, described and disclosed supra can be used in methods for the treatment of hyperplastic tissue growths in subjects by direct injection of the compositions into the hyperplastic tissue growths. Non-limiting examples of hyperplastic tissue growths include benign prostatic hyperplasia (BPH) also known as an enlarged prostate, endometrial hyperplasia, or atypical hyperplasia of the breast. In various embodiments, the hyperplastic tissue is benign prostatic hyperplasia (BPH), endometrial hyperplasia, or atypical hyperplasia of the breast.

In some embodiments, the hyperplastic tissue growth is BPH, wherein the composition is injected directly into the enlarged prostate (intraprostatic injection). The composition can be injected into one or more lobes of the prostate. In some embodiments, the composition is injected into one or more lateral lobes of the prostate. In other embodiments, the composition is injected into the right lateral lobe and the left lateral lobe of the prostate.

The injection of the composition into hyperplastic tissue growths, including enlarged prostates, can be conducted by use of imaging procedures such as “endoscopic ultrasound-guided fine needle injection” (EUS-FNI), which is a procedure in which endoscopy is combined with ultrasound to aid in the location of the hyperplastic tissue growth (or enlarged prostate) and to facilitate the injection of the composition therein. In the case where the composition is a suspension of antineoplastic particles, such as taxane particles, the suspensions of antineoplastic particles can be more easily visualized with sonography than solutions of antineoplastic agents or even suspensions of albumin coated particles. This is especially evident when the antineoplastic particles are in crystalline form. This feature provides a tremendous advantage in that the flow and volume of the suspension can easily be seen during the procedure and can be regulated by the administrator. Thus, in another aspect of the invention, disclosed are methods of administering a suspension composition comprising a carrier and antineoplastic particles to hyperplastic tissue of a subject, the method comprising injecting the composition using endoscopic ultrasound guided-fine needle injection.

The hyperplastic tissue growth is successfully treated when the growth is reduced in volume/size or has reduced growth rate. BPH is successfully treated when the prostate is reduced in volume/size or has reduced growth rate, and/or the subject experiences an improvement of urinary problems associated with BPH.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Example 1. Phase 2 Study—Intraprostatic Injection of Paclitaxel Particles in Subjects With Benign Prostatic Hyperplasia (BPH)

### Summary

In this open-label, dose rising, Phase 2 trial with an expanded cohort at the dose of paclitaxel particles determined to have the best tolerability and safety profile, subjects with benign prostatic hyperplasia (BPH) will have a suspension composition of paclitaxel particles injected under image guidance directly into the right and left lateral lobes of the prostate. The study will include a dose escalation phase and a dose confirmation phase.

In the dose escalation phase, compositions with concentrations of paclitaxel particles of 6, 10, and 15 mg/mL in an injection volume of 10% of the prostate will be studied in cohorts of three, with cohorts enrolled sequentially starting at the lowest concentration. Following Data Safety Monitoring Board (DSMB) review of the cohort data, the next cohort may begin enrolling, an additional three at the current dose may be enrolled, or if the first dose does not provide adequate safety and tolerability, the study may be halted. The dose determined to be the most suitable for further evaluation, defined as the highest dose with an acceptable safety and tolerability profile as determined by the DSMB, will enroll additional subjects to provide a cohort of 12 subjects at that dose level.

Pharmacokinetic samples, PSA, and ejaculate will be collected on regular intervals post injection. Imaging with multiparametric MRI (mpMRI) will be performed two to three weeks prior to treatment by injection of the composition of paclitaxel particles.

### Objectives

The primary objective is to evaluate the safety and tolerability of paclitaxel particles injected directly into the prostate of a subject with benign prostatic hyperplasia. The secondary objectives are: (a) to describe the pharmacokinetics (PK) of paclitaxel particles injected directly into the prostate of a subject with benign prostatic hyperplasia; and (b) to determine the effect of paclitaxel particles on reducing the prostate volume in a subject with benign prostatic hyperplasia.

### Endpoints

The primary endpoint will be safety and tolerability, as assessed by adverse event (AE), changes in laboratory assessments, physical examination findings, and vital signs. The secondary endpoint will be the concentration of paclitaxel in the systemic circulation post-injection.

### Population

Up to a maximum of 30 men at a single/multiple site(s) with benign prostatic hyperplasia.

### Description of Study Agent

Sterile paclitaxel particulate powder for suspension for direct injection into the prostate of a subject with benign prostatic hyperplasia at concentrations of 6, 10, and 15 mg/mL in an injection volume of 10% of the prostate.

1. A method of treating benign prostatic hyperplasia (BPH) in a subject, the method comprising injecting a composition comprising a carrier and an effective amount of a taxane directly into the prostate of the subject, thereby treating the BPH.

2. The method of claim 1, wherein the carrier is a liquid carrier.

3. The method of claim 1, wherein the carrier is an aqueous carrier.

4. The method of claim 1, wherein the taxane is dissolved in the carrier.

5. The method of claim 4, wherein the taxane is paclitaxel or docetaxel.

6. The method of claim 1, wherein the taxane is the form of particles suspended in the carrier.

7. The method of claim 6, wherein the taxane particles have a mean particle size (number) of from 0.1 microns to 5 microns.

8. The method of claim 6, wherein the taxane particles are paclitaxel particles or docetaxel particles.

9. The method of claim 8, wherein the paclitaxel particles or docetaxel particles have a specific surface area (SSA) of at least 18 m²/g.

10. (canceled)

11. The method of claim 8, wherein the paclitaxel particles or docetaxel particles have a bulk density (not-tapped) of 0.05 g/cm³ to 0.15 g/cm³.

12. (canceled)

13. The method of claim 8, wherein the concentration of paclitaxel particles or docetaxel particles in the composition is between about 6 mg/mL and about 15 mg/mL.

14. The method of claim 1, wherein the composition is injected into at least one lateral lobe of the prostate.

15. The method of claim 14, wherein the composition is injected into the left lateral lobe and the right lateral lobe of the prostate.

16. The method of claim 1, wherein the injection of the composition is conducted by endoscopic ultrasound-guided fine needle injection (EUS-FNI).

17. The method of claim 1, wherein the prostate is reduced in volume/size or has a reduced growth rate after injecting the composition; or the subject experiences an improvement in urinary problems associated with BPH after injecting the composition.

18-28. (canceled)
